(19) United States
(12) Patent Application Publication
(10) Pub. No.: US 2025/0352455 A1
(43) Pub. Date: Nov. 20, 2025

(54) PERSONAL CARE VAPOR COMPOSITION

(71) Applicant: Fridababy, LLC, Miami, FL (US)

(72) Inventors: Janette VILLALOBOS, Miami, FL (US); Megan RUMLEY, Miami, FL (US); Chelsea HIRSCHHORN, Miami, FL (US)

(73) Assignee: Fridababy, LLC, Miami, FL (US)

(21) Appl. No.: 18/667,746

(22) Filed: May 17, 2024

(51) Int. Cl. A61K 8/92; A61K 8/34; A61K 8/37; A61K 8/9789; A61Q 13/00; D06F 35/00

(52) U.S. Cl. CPC A61K 8/922; A61K 8/345; A61K 8/375; A61K 8/9789; A61Q 13/00; D06F 35/006

(57) ABSTRACT

A personal care vapor composition, particularly useful for application to the skin of infants, is an emulsion of an oil phase and an aqueous phase. The aqueous phase includes one or more C₃-C₆ polyols. The oil phase includes an emollient selected from one or more triglycerides of C₆-C₁₈ fatty acids and isopropyl isostearate, an olfactory component comprising one or more essential oils, and an olfactory component fixative selected from the group consisting of amphiphilic terpene-based polyethers, Commiphora myrhha extract, methyl hydrogenated rosinate and mixtures thereof. The composition may be easily washed out of clothing that it contacts during use, and has a high perception of vapor intensity.

### BACKGROUND OF THE INVENTION

Personal care vapor compositions are routinely used by consumers on the chest, back, and/or throat to provide relief from cough, nasal congestion, chest congestion, and difficulty sleeping associated with respiratory conditions such as the common cold, flu, RSV, croup, and bronchiolitis. Nasal congestion is either due to inflammation and swelling of the nasal passage, or by blockage with mucus. It can be caused by viral infection (e.g. a cold), allergies, cold dry air, or airborne irritants. Symptoms in infants can include difficulty breathing through the nose, noisy breathing, rapid breathing, lethargy, sinus pressure, snoring, and difficulty eating.

Olfactory compositions are volatile chemical components with a molecular weight of <300 Da that humans perceive via the olfactory system. In the olfactory process, the vapor molecules in the air attach to the cilia of olfactory receptors in the olfactory epithelium, located in the nasal cavity. Olfactory components can be released from personal care compositions as vapors, which are inhaled through the nose and can provide the sensation of cooling and relief. Formulating these kinds of products for infants under the age of 2 years presents a difficult challenge as such compositions need to provide a strong sensory experience but nonetheless are safe for infant skin and mucosal membranes.

Compared with adult skin, infant skin is more susceptible to injury and is more sensitive. Specifically, infant skin appears to have a thinner epidermis and stratum corneum (SC) as well as smaller corneocytes at least until the second year of life. The water-handling properties are not fully developed before the end of the first year and infant SC contains more water and less amounts of natural moisturizing factors. Stamatas, G., et. al., “Infant skin physiology and development during the first years of life: a review of recent findings based on in vivo studies”, Int. J. Cosmetic Science 2011 33:17-24. Thus, infant skin is functionally still developing, and the impaired barrier function of infant skin makes it more susceptible to chemical irritation and local or systemic infections compared with adults.

Olfactory compositions with a high perception of vapor intensity have been found to provide consumers with a stronger sensory experience and smell and, thus, can be perceived by the consumer as a stronger, more effective product. Parents are looking for personal care vapor compositions that can provide a stronger sensory experience and smell, particularly for their children less than 2 years of age. However, it is challenging to incorporate a higher level of an olfactory composition or component in any personal care vapor formulation because these ingredients can be irritating, causing pain and/or skin reddening, on infants below 2 years of age. While camphor and menthol can deliver a high perception of vapor intensity to alleviate the symptoms mentioned above, these ingredients should be avoided in personal care vapor compositions for use on infants below 2 years of age. The FDA's published OTC monograph limits the use of camphor and menthol to children 2 years and over, due to toxicity via oral or inhaled routes. See 20 CFR § 341.74 (d) (2) (Jul. 21, 2000).

Personal care vapor compositions on the market today contain olfactory components comprised of fragrances, terpenes, and/or essential oils, both synthetic and natural or naturally-derived. Fragrances and essential oil compositions can be used to deliver a high perception of vapor intensity, and generally have much lower amounts of camphor and menthol. One way to formulate a product with an increased perception of strength is to increase the level of the fragrance or essential oil in the formula. However, at high levels, these compositions may be irritating to infant skin, causing pain and/or skin reddening.

Personal care vapor compositions on the market are typically sold in natural or naturally-derived oil or petrolatum-based chasses. Oil and petrolatum-based compositions are thought to form an internal scaffold into which oils are dispersed. Once the maximum carrying capacity of the chassis is exceeded, droplets can form on the surface of the product. This process is commonly referred to as “bleeding” or “sweating” and may be unacceptable to consumers. Thus, there are limitations to essential oil levels that can be used to increase the perception of vapor intensity due to safety and stability.

Additionally, oil or petrolatum-based formulas can leave greasy residue on the skin, clothes and fabrics. Emulsions can have better aesthetics and can be made non-staining, however increasing water levels in compositions is known to decrease the perception of vapor intensity. In general, olfactory compositions are more soluble in oil or petrolatum-based chasses. This presents a challenge as increasing levels of water decreases solubility which greatly accelerates the release of vapors into the air of the olfactory composition. Oil or petrolatum-based formulas are typically provided in jars due to their rheological properties and thus require an application that leaves a greasy residue on the hands and skin. To address these issues, some personal care vapor compositions on the market today are provided in a stick format; however, they have limitations on size offerings due to the high product cost of the packaging and formula. Additionally, the amount of product that is possible to apply from a solid stick is reduced, thus limiting the amount of olfactory composition that can be released from the formula.

Therefore, there exists a need in the market for a personal care vapor composition with a high perception of vapor intensity that is safe for infant skin, does not have leave a greasy residue on skin, and can be applied directly to the skin in a mess-free format.

### SUMMARY OF THE INVENTION

According to one aspect of the invention, a personal care vapor composition is provided which comprises: (a) at least about 1 percent of a polyol component selected from one or more C₃-C₆ polyols; (b) at least about 20 percent of an emollient selected from one or more triglycerides of C₆-C₁₈ fatty acids, isopropyl isostearate and mixtures thereof; (c) from about 0.5 to about 10 percent of an olfactory component comprising one or more essential oils; and (d) an olfactory component fixative selected from the group consisting of amphiphilic terpene-based polyethers, Commiphora myrrha extract, methyl hydrogenated rosinate and mixtures thereof, wherein the olfactory component fixative is present in the personal care vapor composition at a ratio of about 1:1 to about 1:4 relative to the olfactory component. The composition has water and oil phases, where the oil phase includes the emollient and the olfactory component, and the water phase includes water and the polyol component. In one embodiment, the oil phase makes up at least 30 percent of the composition. Alternatively, the ratio of the water phase to the oil phase by weight may be less than or equal to 1:1.

The polyol component may be selected from the group consisting of glycerin, diglycerin, erythritol, arabitol, xylitol, mannitol, sorbitol, galactitol, fucitol, mannose, inositol, triethyleneglycol, sodium pyrrolidone carboxylic acid (PCA), zinc PCA, propylene glycol, butylene glycol, pentylene glycol and mixtures thereof. The polyol component may be more particularly present in the composition in an amount less than or equal to 50 percent of the composition, or alternatively may be present in the range of about 5 to about 30 percent of the composition.

According to another aspect of the invention, a personal care vapor composition is provided having (a) at least 10 percent of a polyol component selected from one or more water-soluble polyols; (b) water, a ratio of the water to the polyol components being in the range of about 2.0 to about 0.5; (c) at least about 20 percent of an emollient selected from the group consisting of triglycerides of C₆-C₁₈ fatty acids, isopropyl isostearate and mixtures thereof; (d) from about 1 to about 10 percent of one or more essential oils; and (c) an essential oil fixative selected from the group consisting of amphiphilic terpene-based polyethers, Commiphora myrrha extract, methyl hydroxylated rosinate and mixtures thereof, at a ratio relative to the essential oil in the range of about 1:2 to about 1:4.

In the above aspect, the polyol component may be selected from the group consisting of glycerin, diglycerin, propylene glycol, butylene glycol, pentylene glycol, polyhydric alcohols, 1,3 butylene glycol, polyethylene glycol ethers of glycerin, glycerol monopropoxylate, glycogen, hexylene glycol, hydrogenated starch hydrolysates, hydrolyzed mucopolysaccharides, glycosaminoglycans, methoxy PEG-10, methyl gluceth-10, methyl gluceth-20, methyl glucose, 3-methyl 1,3 butanediol, polyethylene glycol, PEG-15 butanediol, PEG-4, PEG-5, pentaerythritol, PEG-6, PEG-8, PEG-9, 1,2 pentanediol, PPG-1 glyceryl ether, PPG-9, 2-pyrrolidone-5-carboxylic acid and salts thereof such as glyceryl PCA, saccharide isomerate, sericin, silk amino acids, sodium acetylhyaluronate, sodium hyaluronate, sodium polyaspartate, sodium polyglutatmate, sorbet 20, sorbet 6, glucose, mannose, polyglycerol, sorbitol, trehalose, triglycerol, trimethoylpropane, tris (hydroxymethyl) amino methane salts, yeast extract and mixtures thereof. The polyol component may be present in the composition in the range from about 5 to about 30 percent.

In either of the above aspects, the emollient may be selected from the group consisting of isopropyl isostearate, caprylic/capric triglycerides, one or more vegetable oils such as hydrogenated vegetable oil, olive oil, coconut oil, sunflower oil and mixtures thereof. The emollient may be present in the composition at no more than 70 percent, and more particularly may be present in the range of about 30 to about 70 percent.

In either of the above aspects, the olfactory component or one or more essential oils may be selected from the group consisting of eucalyptus oil, lavender oil, rosemary oil, spearmint oil, wintergreen oil, cedar leaf oil, levomenthol when present as a component of an essential oil blend, cedar wood oil, turpentine, thymol, anethole, coriander, mandarin, petitgrain, armoise, cumin nitrile, marjoram sweet, pink pepper, basil, frankincense, methyl salicylate, bergamot, galbanum, neroli, Sichuan pepper, black pepper, grapefruit, nutmeg oil, tea tree oil, cardamom, jasmine, oil of black pepper, verveine, Chinese ginger oil, orange sweet, vetivert, cinnamon leaf, lavendine, palmerosa, violet leaves, clary sage, lemongrass, patchouli oil, ylang, clove, lime, olbus oil and mixtures thereof. The olfactorycomponent or one or more essential oils more particularly may be present in the composition at about 2 to about 4 percent.

In a personal care vapor composition formulated particularly for infants, the olfactory component may be selected from the group consisting of eucalyptus oil, lavender oil, rosemary oil, wintergreen oil, spearmint oil and mixtures thereof.

In either of the above aspects, the essential oil fixative or olfactory component fixative may be polycitronellol.

In either of the above aspects, the personal care vapor composition may further include from about 0.01% to about 15% of a structuring agent or structurant selected from the group consisting of glyceryl stearate, stearic acid, palmitic acid, stearyl alcohol, cetyl alcohol, behenyl alcohol, one or more polyethylene glycol ethers of stearyl alcohol having an average of about 1 to about 5 ethylene oxide units, one or more polyethylene glycol ethers of cetyl alcohol having an average of about 1 to about 5 ethylene oxide units, and mixtures thereof.

In either of the above aspects, the personal vapor care composition may further include from about 0.05% to about 5.0% of an emulsifier selected from the group consisting of fatty phosphate monoesters, fatty acid sucrose esters, phospholipids, fatty acid mono- and diglycerides, ethoxylated alcohols, polysorbates and mixtures thereof. Examples of such emulsifiers are potassium cetyl phosphate, sucrose laurate, sucrose palmitate and sucrose stearate.

According to another aspect of the invention, a method is provided for soothing cough, nasal congestion, chest congestion, and difficulty sleeping associated with respiratory conditions such as the common cold, flu, RSV, croup, or bronchiolitis in infants. The method may comprise the steps of: a) providing a personal care vapor composition; and b) applying a safe and effective amount of the personal care vapor composition to at least a portion of a user's chest, back, and/or neck in need thereof. The personal care vapor composition may comprise a polyol component comprising one or more water-soluble polyols, an emollient selected from the group consisting of one or more triglycerides of C₆-C₁₈ fatty acids, isopropyl isostearate and mixtures thereof, an olfactory component comprising one or more essential oils, an essential oil fixative, and a dermatologically acceptable carrier such as water. The composition may further include a structurant.

According to a further aspect of the invention, a method is provided for using a personal care vapor composition. The method includes the steps of (a) supplying a personal care vapor composition comprising water, one or more water-soluble polyols, one or more triglycerides of C₆-C₁₈ fatty acids, an olfactory component, and an olfactory component fixative; (b) applying the personal care vapor composition to the skin of a human; (c) contacting a fabric to the skin of the human in an area where the personal care vapor composition has been applied, whereby some of the personal care vapor composition adheres to the fabric; (d) removing the fabric from the human; (e) washing the fabric in a washing machine using cold water and a detergent; and (f) responsive to the step of washing, completely removing the personal care vapor composition from the fabric. It has been found that personal care vapor compositions made according to the invention wash out of common fabrics without leaving a greasy residue, greatly enhancing their acceptability to caregivers.

### BRIEF DESCRIPTION OF THE DRAWINGS

Further aspects of the invention and their advantages may be discerned in the following detailed description, when read in conjunction of the drawings, in which:

FIG. 1 is a flow chart depicting steps in a method for using a personal care vapor composition;

FIGS. 2A-2E are photographs of flags of five different fabrics, after a composition according to the invention has been applied to them, and after they have been washed and dried;

FIGS. 3A-3E are photographs of flags of the same fabrics as those shown in FIGS. 2A-2E, after a first prior art commercial personal care vapor composition has been applied to them, and after they have been washed and dried; and

FIGS. 4A-4E are photographs of flags of the same fabrics as those shown in FIGS. 2A-2E, and FIGS. 3A-3E, after a second prior art commercial personal vapor composition has been applied to them, and after they have been washed and dried.

### DETAILED DESCRIPTION OF THE INVENTION

As used herein, the term “derivative” refers to structures which are not shown but which one skilled in the art would understand are variations of the basic compound.

As used herein, “skin care” means regulating and/or improving a skin condition. Some nonlimiting examples include: improving skin appearance and/or feel by providing a smoother, more even appearance and/or feel; increasing the thickness of one or more layers of the skin; improving the elasticity or resiliency of the skin; improving the firmness of the skin; reducing the oily, shiny, and/or dull appearance of skin; improving the hydration status or moisturization of the skin; improving the appearance of fine lines and/or wrinkles; improving skin exfoliation or desquamation; plumping the skin; improving skin barrier properties; improve skin tone; reducing the appearance of redness or skin blotches; soothing irritated skin feel and/or improving the brightness, radiancy, or translucency of skin; preventing damage to skin via antioxidant approaches, including UV A and UV B induced damage; preventing formation of comedones, balancing the skin microbiome; and preventing acne.

As used herein as a noun, “active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

As used herein, the term “personal care topical application” means to apply or spread the compositions of the present invention onto the surface of the skin.

The phrase “dermatologically acceptable carrier”, as used herein, means that the carrier is suitable for personal care topical application to the keratinous tissue without undue toxicity, incompatibility, instability, allergic response, and the like, has good aesthetic properties, and is compatible with the other components of the personal care vapor composition. Dermatologically acceptable carriers include purified water. Other dermatologically acceptable carriers, such as sunflower oil, may be in an oil phase of the composition.

As used herein, “olfactory component” refers to a composition comprising odoriferous material(s) which are able to provide a fragrance. The odoriferous materials can be olfactory agents and can include essential oils, aromatic oils, excipients, actives and/or perfumes.

As used herein, the term “high perception of vapor intensity” means a strong sensory experience and smell that consumers perceive as strong and effective.

As used herein, the terms “olfactory component fixative” and “essential oil fixative” denoted a class of ingredients that enhance the high perception of vapor intensity of the personal care vapor composition.

As used herein, the terms “plant extract” or “natural extract” or “extract” with reference to a plant is any material extracted from natural resources including plants. The entire plant or any part of the plant including the bark, berries, flowers, leaves, stem, stalk, peels, resins, rhizome, roots, seeds, woods and mixtures thereof may be used for the extraction process. Extracts may be obtained using any suitable method known in the art including: milling, grinding, maceration, infusion, percolation and decoction, Soxhlet extraction, microwave assisted extraction, ultra-sound-assisted extraction, sonication extraction, solvent extraction, accelerated solvent extraction, and supercritical fluid extraction. Suitable extraction solvents may include water, ketones, esters, C₁ to C₆ alcohols, hydrocarbons and mixtures thereof.

As used herein, the term “safe and effective amount” means an amount of a material, ingredient, compound, component, or composition sufficient to significantly induce a positive benefit, but low enough to avoid serious side effects such as undue toxicity or allergic reaction.

As used herein, the term “skin” means the outermost protective covering of mammals that is composed of cells such as keratinocytes, fibroblasts and melanocytes. Skin includes an outer epidermal layer and an underlying dermal layer. Skin may also include hair follicles, sebaceous gland and nails as well as other types of cells commonly associated with skin, such as, for example, myocytes, Merkel cells, Langerhans cells, macrophages, stem cells, sebocytes, nerve cells and adipocytes.

As used herein, “ambient conditions” refers to a temperature of about 23 degrees Celsius (° C.) and 50% relative humidity (RH).

As used herein, “viscosity” means the viscosity at about 0.10 s⁻¹ as determined by the Rheology Test Method, described hereafter.

As used herein, “Brookfield viscosity” means the viscosity as determined by the Brookfield Viscosity Test Method, described hereafter.

As used herein, the articles “a” and “an” are understood to mean one or more of the materials that is claimed or described, for example, “a rheology modifier” or “an active.”

As used herein, the terms “include,” “includes,” and “including” are meant to be nonlimiting.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (C) unless otherwise indicated. Unless otherwise specified, all measurements herein are conducted at 20° C., under atmospheric pressure, and at 50% relative humidity.

In the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Active and other ingredients useful herein may be categorized or described herein by their cosmetic, olfactory and/or therapeutic benefit or their postulated mode of action. However, it is to be understood that the active and other ingredients useful herein can in some instances provide more than one cosmetic, olfactory and/or therapeutic benefit or operate via more than one mode of action. Therefore, classifications herein are made for the sake of convenience and are not intended to limit an ingredient to the particularly stated application or applications listed.

All weights, measurements and concentrations herein are measured at ambient conditions unless otherwise specified.

The compositions of the present invention can comprise, consist essentially of, or consist of, the claimed components as well as optional ingredients described herein. As used herein, “consisting essentially of” means that the composition or component may include additional ingredients, but only if the additional ingredients do not materially alter the basic and novel characteristics of the claimed compositions or methods.

Personal care vapor compositions of the present invention may be applied to mammalian keratinous tissue, in particular to human skin. The personal care vapor compositions disclosed herein may be used on the chest, back, and/or neck to provide relief from cough, nasal congestion, chest congestion, and difficulty sleeping associated with respiratory conditions such as the common cold, flu, RSV, croup, or bronchiolitis. Olfactory components can be released from personal care compositions as vapors, which are inhaled through the nose and can provide the sensation of cooling and relief. The personal care vapor compositions according to the invention may be made into a wide variety of product forms. These may include, but are not limited to, creams, lotions, serums, sprays, tonics, gels, solutions, suspensions, aerosol sprays, sticks, ointments, liquid washes, soap bars, shampoos, hair conditioners, pastes, foams, powders, mousses, shaving creams, hydrogels, film-forming products, and the like. The personal care vapor composition form may follow from the particular dermatologically acceptable carrier chosen.

The personal care vapor compositions can be a stable emulsion with a continuous aqueous phase (e.g., an oil-in-water or water-in-oil-in-water emulsion) or a continuous oil phase (e.g., water-in-oil or oil-in-water-in-oil emulsion) and a dispersed phase. An emulsifier can promote the formation of the emulsion and can also help maintain product stability.

Petrolatum (e.g. Vick's®) does not absorb into skin and leaves a greasy film on skin that was found to stick to and stain clothing. Incorporation of water in the formula (e.g. through an emulsion) was found to enable the product to absorb into instead of sitting on top of skin, allowing for a non-sticky, mess free experience. Additionally, to create a formula compatible with a hands-free applicator, the inclusion of water and/or a polyol is required. Generally, the inclusion of water and/or glycerin tends to lower the perception of scent intensity. Thus, the incorporation of water led to a tradeoff in the initial perception of vapor intensity and long-lasting scent compared to compositions using petrolatum as a base. One way to formulate a product with an increased perception of strength is to increase the level of essential oil in the formula. However, as the essential oil concentration increases, physical stability decreases, which can affect both the appearance of the final product as well as the distribution of the essential oil within the composition. Olfactory composition fixative technologies are known and are typically designed for use in oil compositions such as fragrances and can contribute to product instability in an emulsion.

Surprisingly it was found that a combination of high levels of an emollient or emollient blend, a structurant, a polyol, and an amphiphilic, polymeric-based olfactory component fixative allowed for the incorporation of essential oils at a safe level for infant skin with a high perception of vapor intensity that was non-staining on fabric and that had a non-greasy feel on skin. Without being bound by theory, it is believed that the terpene-based polyether olfactory component fixative has sufficient hydrolytic stability to be compatible in an emulsion and solubilizes the olfactory component in the personal care vapor composition to partition the controlled release of the vapors over an extended period of time.

In some examples, the personal care vapor composition can be a moisturizer with a viscosity that is sufficient for it to be dispensed from a pump, through a container that is squeezed (e.g., a tube) without sloshing or easily spilling, or from a hands free container such as a tube with a built in applicator and is also thin enough so it can be easily spread across a user's skin including but not limited to the chest, back, throat and/or other portions of the body. The viscosity at about 0.10 s⁻¹ can be greater than or equal to 550 Pas, alternatively greater than or equal to 600 Pa*s, alternatively greater than or equal to 1500 Pas, alternatively greater than or equal to 2000 Pa*s, alternatively greater than or equal to 2500 Pas, alternatively greater than or equal to 3000 Pa*s. The average viscosity can be from about 550 Pa*s to about 6000 Pa*s, alternatively from about 600 Pa*s to about 5500 Pa*s, alternatively from about 800 Pa*s to about 5000 Pa*s, and alternatively from about 1000 Pa*s to about 3000 Pa*s. The viscosity at about 0.10 s¹ can be determined by the Rheology Test Method, described hereafter.

### Olfactory Component

Personal care vapor compositions according to the invention have an olfactory component comprising one or more olfactory agents. Non-limiting examples of olfactory agents can include eucalyptus oil, rosemary oil, cedar leaf oil, levomenthol (when present in a blend with other essential oils), cedar wood oil, turpentine, thymol, anethole, coriander, mandarin, petitgrain, armoise, cumin nitrile, marjoram sweet, pink pepper, basil, frankincense, methyl salicylate, bergamot, galbanum, neroli, sichuan pepper, black pepper, grapefruit, nutmeg oil, tea tree oil, cardamom, jasmine, oil of black pepper, verveine, chinese ginger oil, lavender oil, orange sweet, vetivert, cinnamon leaf, lavendine, palmarosa, violet leaves, clary sage, lemongrass, patchouli oil, ylang, clove, lime, spearmint oil, wintergreen oil, olbus oil, and combinations thereof. Of these, an olfactory component particularly suitable for a personal care vapor composition meant to be applied to the skin of an infant may include eucalyptus oil, rosemary oil, lavender oil, spearmint oil, wintergreen oil and mixtures thereof.

In some embodiments, the olfactory component or essential oil can be selected from the group consisting of eucalyptus oil, lavender oil, rosemary oil, peppermint oil and mixtures thereof.

The personal care vapor composition can comprise from about 0.05% to about 10% of an olfactory component, alternatively from about 2% to about 8%, alternatively from about 3% to about 5%, alternatively about 4%. It was surprisingly found that personal care vapor compositions with less than about 10% of an olfactory component had a high perception of vapor intensity.

### Olfactory Component Fixative

Personal care vapor compositions according to the invention also comprise from about 1% to about 4%, preferably from about 1% to about 2%, more preferably about 1.5%, of an olfactory component fixative or essential oil fixative. The olfactory component fixative may be selected from the group consisting of polymeric amphiphilic molecules such as methyl hydrogenated rosinate, Commiphora myrrha resin extract, amphiphilic terpene-based polyethers and mixtures thereof. In embodiments, the olfactory component fixative may be polycitronellol. The olfactory component fixative may be present in the composition at a ratio of about 1:1 to about 1:4, alternatively about 1:2 to about 1:4, alternatively about 3:8, relative to the olfactory component.

### Polyol Component

Personal care vapor compositions according to the invention also comprise from about 1% to about 50%, preferably from about 5% to about 30%, more preferably from about 15% to about 30%, of a polyol component having one or more polyols. Polyols are substances which provide the skin with water-retention benefits. Polyols have an affinity to hydrogen bonds of water molecules and to skin hydrophilic molecular functionalities, and accordingly bind themselves to internal water molecules as well as skin molecules, holding water to the outside surface and upper layers of the stratum corneum, thereby increasing the overall content in the skin itself. Topical application of compositions containing polyols, for example glycerin, can be associated with improvements in barrier function, increases in epidermal thickness, and improvements in general skin appearance.

The polyol component of the personal care vapor composition may comprise one or polyols selected from the group consisting of polyhydric alcohols, butylene glycol, propylene glycol, pentylene glycol, glycerin, diglycerin, polyethylene glycol ethers of glycerin (e.g., glycereth 20), glycerin, glycerol monopropoxylate, glycogen, hexylene glycol and extracts or derivatives thereof, hydrogenated starch hydrolysates, hydrolyzed mucopolysaccharides, inositol, glycosaminoglycans, methoxy PEG-10, methyl gluceth-10, methyl gluceth-20, methyl glucose, 3-methyl-1,3-butanediol, polyethylene glycol and derivatives thereof (such as PEG-15 butanediol, PEG-4, PEG-5 pentaerythritol, PEG-6, PEG-8, PEG-9), pentaerythritol, 1,2 pentanediol, PPG-1 glyceryl ether, PPG-9, 2-pyrrolidone-5-carboxylic acid and its salts such as glyceryl PCA, saccharide isomerate, sericin, silk amino acids, sodium acetylhyaluronate, sodium hyaluronate, sodium polyaspartate, sodium polyglutamate, sorbeth 20, sorbeth 6, sugar and sugar alcohols and derivatives thereof such as glucose, mannose and polyglycerol sorbitol, trehalose, triglycerol, trimethyolpropane, tris (hydroxymethyl) amino methane salts, yeast extract, and mixtures thereof.

Non-limiting examples of polyhydric alcohols not specifically listed above include C₃-C₆ polyols such as erythritol, arabitol, xylitol, ribitol, mannitol, sorbitol, galactitol, fucitol, maltitol, mannose, inositol, triethyleneglycol, sodium pyrrolidone carboxylic acid (PCA), zinc PCA and derivatives and mixtures thereof.

### Emollients

Personal care vapor compositions according to the invention comprise from about 20% to about 70%, preferably from about 30% to about 70%, more preferably from about 35% to about 45%, of an emollient. Emollients tend to lubricate the skin, increase the smoothness and suppleness of the skin, prevent or relieve dryness of the skin, and/or protect the skin. Emollients may physically prevent or reduce moisture loss from the skin by formation of a water-impenetrable barrier over the stratum corneum. Emollients are typically water-immiscible, oily or waxy materials. The level of emollient within the personal care vapor composition may vary according to the form of the personal care vapor composition. Nonlimiting examples of emollients include isopropyl isostearate and triglycerides of C₆-C₁₈ fatty acids such as caprylic/capric triglycerides, vegetable oils such as hydrogenated vegetable oil, coconut oil, olive oil, almond oil, avocado oil, sunflower oil and mixtures thereof. The personal care vapor composition more particularly may comprise an emollient selected from the group consisting of isopropyl isostearate, caprylic/capric triglycerides, hydrogenated vegetable oil, olive oil, coconut oil, sunflower oil and mixtures thereof.

### Structuring Agent

Personal care vapor compositions according to the invention may comprise from about 0.01% to about 15%, preferably from about 0.1% to about 10%, more preferably from about 0.5% to about 5%, of a structuring agent or structurant. Structuring agents may be particularly preferred in oil-in-water emulsions. Without being limited by theory, it is believed that the structuring agent may assist in providing rheological characteristics to the composition which contribute to the stability of the composition. For example, the structuring agent tends to assist in the formation of the liquid crystalline gel network structures. The structuring agent may also function as an emulsifier or surfactant.

Non-limiting examples of structuring agents include glyceryl stearate, stearic acid, palmitic acid, stearyl alcohol, cetyl alcohol, behenyl alcohol, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. Preferably, the personal care vapor composition may comprise a structuring agent selected from the group consisting of stearic acid, palmitic acid, stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

### Dermatologically Acceptable Carrier

A personal care vapor composition according to the present invention comprises a safe and effective amount, for example, from about 10% to about 45%, preferably from about 15% to about 18%, of a dermatologically acceptable carrier (“carrier”). The carrier can thus act as a diluent, dispersant, solvent, or the like to ensure that the other materials of the composition can be applied to and distributed evenly over the selected target at an appropriate concentration. The carrier may be water, particularly purified water. Where water is used as the carrier, a ratio of the water to the polyol component may be in the range of about 2.0 to about 0.5, and alternatively about 1.8 to about 0.5. Where water is used as the carrier, the composition has an aqueous phase and an oil phase, with the aqueous phase including the water and the polyol component, and the oil phase including the emollient and the olfactory component. In embodiments where water is used as the carrier, the ratio of the aqueous phase to the oil phase may be less than or equal to 1:1, and for example may be in the range of about 0.4 to about 1.0. Alternatively the oil phase of the composition may be at least 30 percent of the composition.

### Emulsifiers

Personal care vapor compositions according to the invention may further comprise one or more emulsifiers. An emulsifier is a substance which aids in the creation of an oil/water or a water/oil emulsion and promotes product stability. The emulsifier may be present in the personal care vapor composition in the range of about 0.05% to about 5%, alternatively about 0.25% to about 0.5%. Suitable emulsifiers include polyglyceryl esters such as candelilla/jojoba/rice bran polyglyceryl-3 esters and polyglyceryl-6 distearate, sucrose esters of fatty acids such as sucrose palmitate and sucrose stearate, phospholipids, fatty acid mono- and diglycerides, polysorbates and mixtures thereof.

### Preservatives

The personal care vapor composition may comprise at least 0.0001%, about 0.001%, about 0.01%, about 0.05%, about 0.1%, about 0.5%, about 1%, or about 2%, of a preservative. The preservative prevents or retards the formation of yeast, bacteria, and/or mold.

The personal care vapor composition of the present invention may comprise a preservative selected from the group consisting benzoic acid and salts thereof, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, sodium benzoate, calcium benzoate, calcium propionate, caprylyl glycol, biguanide derivatives, captan, chlorohexidine diacetate, digluconate, chlorhexidine dihydrochloride, chloroacetamide, chlorobutanol, p-chloro-m-cresol, chlorophene, chlorothymol, chloroxylenol, m-cresol, o-cresol, DEDM hydantoin, DEDM hydantoin dilaurate, dehydroacetic acid, diazolidinyl urea, dibromopropamidine diisethionate, DMDM hydantoin, glyceryl caprylate, potassium sorbate, salicylic acid, hexamidine, capryloyl glycine, 1,2-hexanediol, undecylenoyl glycine, ethylhexylglycerin, caprylhydroxamic acid, methylpropanediol, propanediol, hinokitiol, sodium hinokitiol, phenylethyl alcohol, levulinic acid, p-anisic acid, 2-bromo-2-nitropropane-1,3-diol, sodium hydroxymethylglycinate, iodopropynyl butylcarbamate, methylchloroisothiazolinone, methylisothiazolinone, piroclone olamine, cinnamon oil, rosemary extract, and combinations thereof.

### Additional Optional Components

A wide variety of optional skin care composition components may further be included in the personal care vapor compositions. For example, the personal care vapor composition may comprise a skin care composition component selected from the following absorbents, abrasives, anticaking agents, antifoaming agents, propellants, antimicrobial agents, external analgesics, binders, biological additives, buffering agents, bulking agents, chemical additives, cosmetic biocides, denaturants, cosmetic astringents, drug astringents, film formers, opacifying agents, fragrances, pigments, colorings, skin soothing agents, pH adjusters, plasticizers, preservative enhancers, reducing agents, additional skin-conditioning agents, skin penetration enhancing agents, skin protectants, solvents, suspending agents, solubilizing agents, sunscreens, peptides (e.g., peptides containing ten or fewer amino acids, their derivatives, isomers, and complexes with other species such as metal ions), ultraviolet light absorbers or scattering agents, tanning agents, antioxidants and/or radical scavengers, chelating agents, acute powders, oil/sebum control agents, sweat control agents, sequestrants, anti-acne agents, anti-inflammatory agents, anti-androgens, depilation agents, sugar amines (e.g., N-acetyl-glucosamine), desquamation agents/exfoliants, oil control agents, anti-cellulite actives, skin lightening agents, flavonoids, protease inhibitors (e.g., hexamidine and derivatives), non-vitamin antioxidants and radical scavengers, anti-wrinkle actives, anti-atrophy actives, minerals, phytosterols and/or plant hormones, tyrosinase inhibitors, N-acyl amino acid compounds, moisturizers, organic hydroxy acids, vitamins and derivatives thereof, natural or plant extracts, and mixtures thereof. Suitable actives are further described in U.S. Patent Appln. Pub. Nos. 2006/0275237 A1 to Bissett et al. and 2004/0175347 A1 to Bissett. One skilled in the art will recognize that a particular ingredient may have more than one function.

### Vitamins and Derivatives Thereof

The personal care vapor composition may comprise a safe and effective amount of one or more vitamins and derivatives thereof. Non-limiting examples of vitamins and derivatives thereof include vitamin B3 compounds such as niacinamide, niacinnicotinic acid, tocopheryl nicotinate; vitamin B5 compounds, such as panthenol; vitamin A compounds and natural and/or synthetic analogs of vitamin A, including retinoids, retinal, retinyl acetate, retinyl palmitate, retinoic acid, retinaldehyde, retinyl propionate, carotenoids (provitamin A); vitamin E compounds or tocopherol, including tocopheryl sorbate and tocopheryl acetate; vitamin C compounds, including ascorbate, ascorbyl esters of fatty acids, and ascorbic acid derivatives such as magnesium ascorbyl phosphate and sodium ascorbyl phosphate, ascorbyl glucoside, and ascorbyl sorbate, and derivatives of any of the aforementioned actives.

### Skin Soothing Agents

The personal care vapor composition may comprise a safe and effective amount of a skin soothing agent, preferably from about 0.1% to about 30%, more preferably from about 0.5% to about 20%, more preferably from about 0.5% to about 10%, by weight of the personal care vapor composition. Nonlimiting examples of skin soothing agents suitable for use herein may include panthenoic acid derivatives (including panthenol, dexpanthenol and ethyl panthenol), aloe vera, allantoin, bisabolol, dipotassium glycyrrhizinate, pentylene glycol, 4-t-butylcyclohexanol, PEG-40 hydrogenated castor oil, hydroxyphenyl propamidobenzoic acid, trideceth-9, propylene glycol, Zingiber officinale (ginger) root extract, and mixtures thereof. Nonlimiting examples of of suitable commercially available preservatives include SymSitive® (pentylene glycol and 4-t-butylcyclohexanol); SymCare® (pentylene glycol, 4-t-butylcyclohexanol, PEG-40 hydrogenated castor oil, trideceth-9, hydroxyphenyl propamidobenzoic acid and propylene glycol); and SymRelief® (bisabolol and Zingiber officinale (ginger) root extract), all made available by Symrise of Holzminden, Germany.

### Chelators or Chelating Agents

The personal care vapor composition may comprise a safe and effective amount of a chelator or chelating agent, preferably from about 0.1% to about 10%, more preferably from about 1% to about 5% of the personal care vapor composition. As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation which can contribute to excessive scaling or skin texture changes and against other environmental agents which can cause skin damage. An example of a chelating agent is disodium EDTA.

### Viscosity and pH of the Personal Care Vapor Composition

The viscosity and pH of the personal care vapor composition of the present invention may depend on the type or form of product desired for the composition. Generally, the personal care vapor composition may have a viscosity of from about 30,000 cP to about 300,000 cP when measured according to the Viscosity Test Method described herein. The viscosity of the composition as used herein is described as the Brookfield viscosity.

Nonlimiting exemplary personal care vapor compositions of the present invention wherein the composition is in the form of a lotion may have a viscosity of from about 30,000 cP to about 100,000 cP. Nonlimiting exemplary personal care vapor compositions of the present invention wherein the composition is in the form of a cream may have a viscosity of from about 50,000 cP to about 300,000 cP. Nonlimiting exemplary personal care vapor compositions of the present invention wherein the composition is in the form of a serum may have a viscosity of from about 10,000 cP to about 30,000 cP.

The pH values of these compositions preferably range from about 2 to about 8, more preferably from about 3 to about 7, even more preferably from about 4 to about 6. pH is measured according to the pH Test Method as described herein.

### Methods of Making Personal Care Vapor Compositions

The personal care vapor compositions of the present invention may be generally prepared by conventional methods such as are known in the art of making personal care vapor compositions. Such methods typically involve mixing of the ingredients in one or more steps to a relatively uniform state, with or without heating, cooling, application of vacuum, and the like. Typically, emulsions may be prepared by first mixing the aqueous phase materials separately from the fatty or oily phase materials and then combining the two phases as appropriate to yield the desired continuous phase. The compositions may preferably be prepared such as to optimize stability (e.g., physical stability, chemical stability) and/or delivery of the active materials.

### Methods of Using Personal Care Vapor Compositions

Personal care vapor compositions of the present invention may be used to treat symptoms of congestion. The treatment method may include the steps of providing a personal care vapor composition according to the present invention and applying a safe and effective amount of the personal care vapor composition to at least a portion of a subject's skin, neck, and/or back in need thereof.

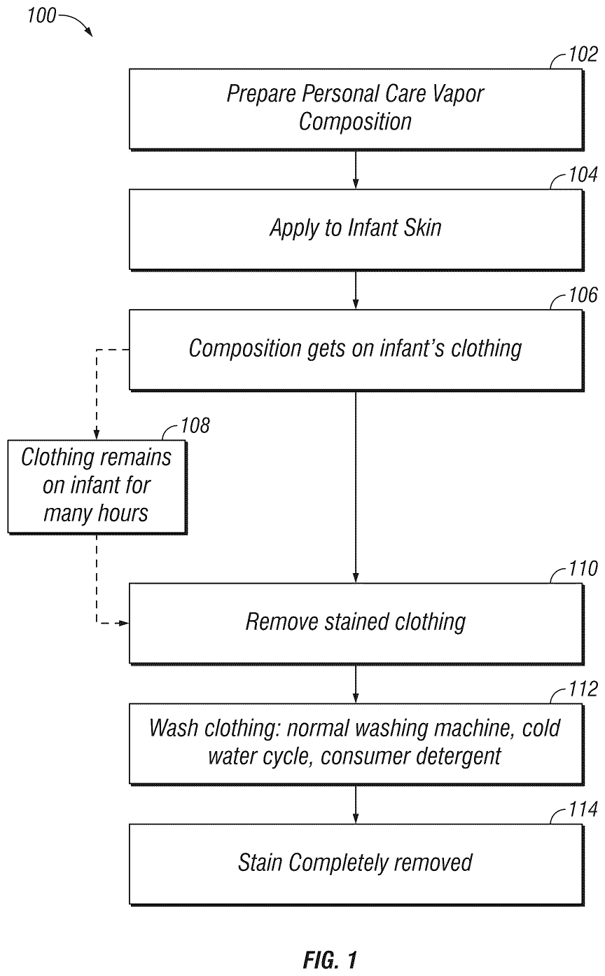

A further method of use (100) is illustrated in FIG. 1. At step 102, a personal care vapor composition is formulated with the components disclosed herein. At step 104, the composition is applied to the skin of an infant, such as on the chest. The infant may be dressed in one or more articles of clothing. As a result of the topical application of the composition, some of the composition may adhere to such an article of clothing at step 106. Possibly, and as shown at step 108, the composition may remain in contact with the clothing for some hours.

At step 110, the caregiver undresses the infant and removes the stained article of clothing. At step 112, the article is put through a normal cold-water wash in a normal washing machine using a consumer detergent. As a result of this normal washing cycle, and surprisingly, the composition is completely removed (step 114) from the article of clothing.

### Stain Test Method

The superior washability of personal care vapor compositions according to the invention was demonstrated by the following stain test method. Fabrics were cut into 6×6 in. squares to create swatches or flags. Each flag was labelled with a sharpie to identify the test product that was to be applied to it. A typical usage amount of product is weighed and applied in a thin layer as evenly as possible to each fabric flag. The product was allowed to set on the fabric for 8 hours. Next, fabric flags, white towel ballast, and ALL® Free Clear Original liquid detergent were added to a washing machine. The amount of detergent used was as directed by the manufacturer (Henkel Corporation). A standard cold water cycle was run. At the completion of the washer cycle, the flags and ballast were removed from the washing machine and placed in a dryer. The dryer was run at a standard high temperature cycle until the flags were completely dry. The washed and dried test flags were then photographed.

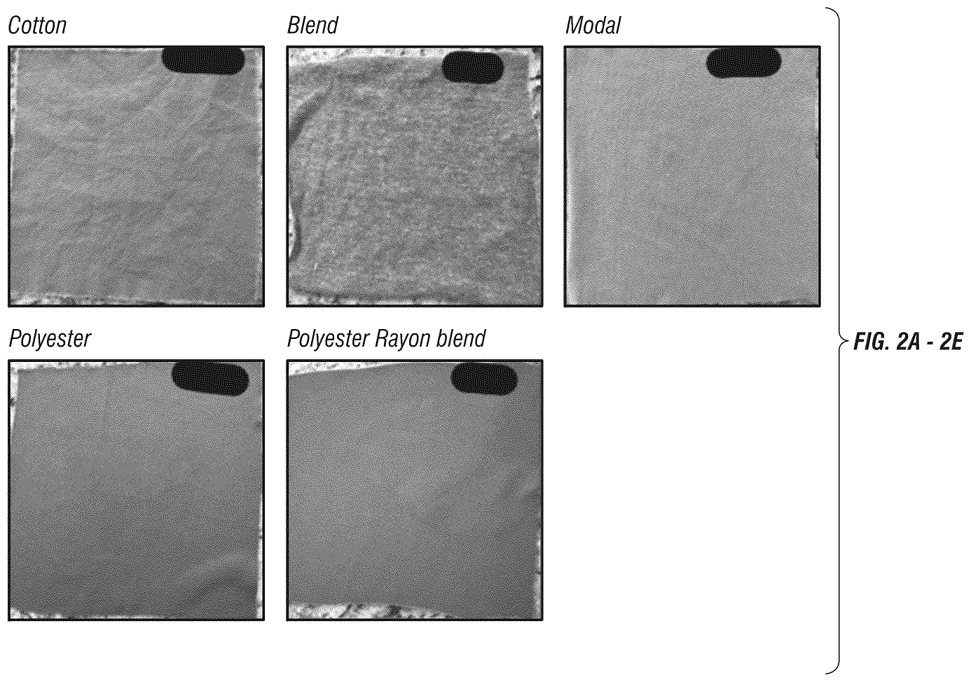

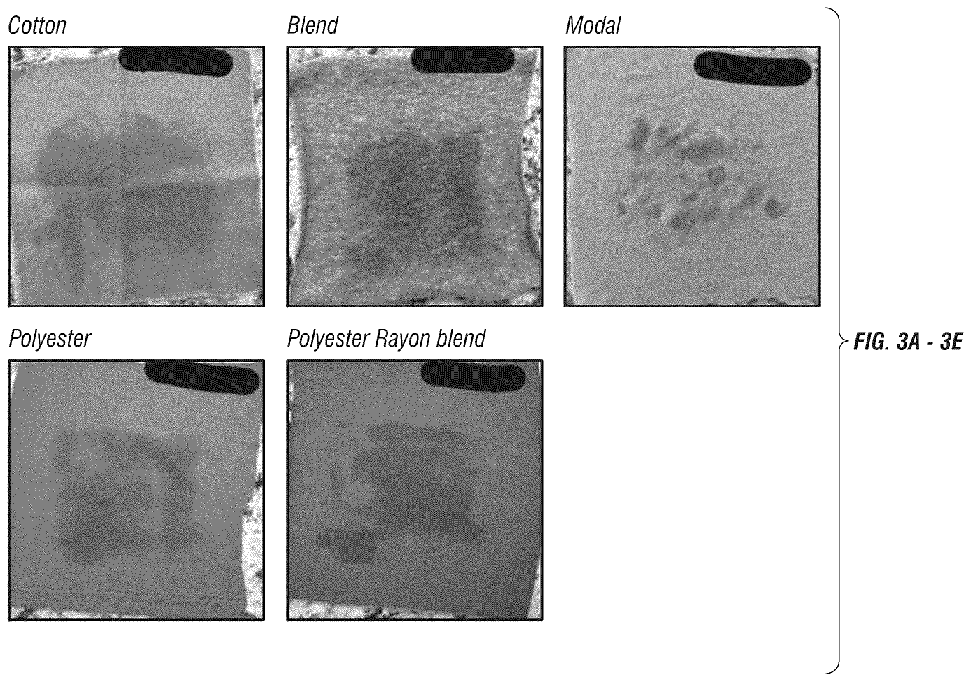

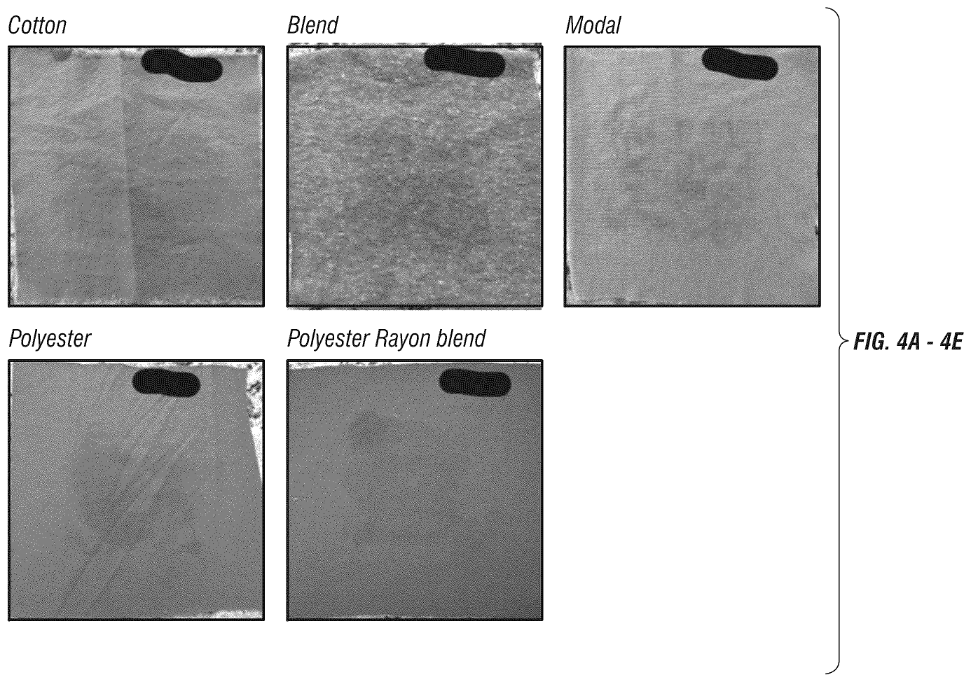

FIGS. 2A-2E are photographs of test flags to which “Exemplary Composition 1”, as specified in Table 1, had been applied. The flags include cotton (FIG. 2A), blend (50% polyester, 40% cotton, 10% rayon; FIG. 2B), Modal (a semi-synthetic textile made from beechwood cellulose, FIG. 2C), polyester (FIG. 2D) and polyester rayon blend (FIG. 2E). A prior art composition called “Comparison 1”, as specified in Table 1, was applied to the flags shown in FIGS. 3A-3E, which were made of respective materials identical to those of FIGS. 2A-2E. A prior art composition called “Comparison 2”, as specified in Table 1, was applied to the flags shown in FIGS. 4A-4E, which also were made of respective materials identical to those in FIGS. 2A-2E. All test flags were stained, washed and dried according to the Stain Test Method described above. As can be seen, “Exemplary Composition 1” was completely removed from all fabrics. But this was not the case for “Comparison 1” or “Comparison 2”, for both of which significant staining persisted.

### Viscosity and pH Test Methods

### Brookfield Viscosity Test Method

The viscosity of samples can be measured using a standard viscometer, such as, for example, a Brookfield DV2T viscometer (manufactured by Brookfield Ametek, Middleboro, MA, US), fitted with a helipath T-bar spindle type T-C. The viscometer is leveled, set up and calibrated according to the manufacturer's standards. The viscometer speed (RPM) is selected to ensure the measured viscosity is within the manufacturer's recommended settings (e.g., 5 RPM).

Samples are stored in sealed glass jars with an opening and internal diameter of at least 40 mm and filled to a height of at least 50 mm with care taken to avoid entrapped air bubbles. Centrifugation may be used to help removed entrained air. Sample jars are equilibrated at 23° C.±2° C. and about 50%±2% relative humidity for at least 24 hours prior to measurement.

Viscosity is measured at 23° C.±2° C. and about 50%±2% relative humidity by placing the uncapped sample jar under the viscometer and lowering the viscometer until the tip of the T-bar touches the surface of the sample. The descending helipath is turned on and a timer started once the cross-bar of the T-bar touches the surface of the sample. A reading is taken about every 10 seconds over the time period of between about 45 seconds and about 1 minute. The viscosity is calculated as the arithmetic average of the viscosities recorded. Care is taken to ensure the T-bar does not touch the glass jar.

### pH Test Method

pH can be measured using a standard pH meter such as, for example, a Beckman Coulter model PHIi 410 pH meter equipped with a general-purpose probe (manufactured by Beckman Coulter, Brea, CA, US). The pH meter is calibrated according to the manufacturer's instructions. Measurements are performed after equilibrating the compositions to room temperature (approximately 23° C.±2° C.)

### Examples

The following data and examples, including comparative examples, are provided to help illustrate personal care vapor compositions, are given solely for the purpose of illustration, and are not to be construed as limitations of the present invention, as many variations thereof are possible without departing from the spirit and scope of the invention.

Compositions shown below in Table 1 were prepared according to the procedure described below.

Phase A was prepared by adding all of the Phase A ingredients to a beaker and mixing while heating to 70-85 C. Phase B was prepared by combining all of the Phase B ingredients in a separate beaker equipped with a stir bar and heating the ingredients to between about 70° C. and about 85° C. The following took place under continuous mixing using an overhead mixer. Phase B was poured into Phase A very slowly and the resulting mixture was mixed under high shear. The mixture was then cooled to between about 55° C. and about 65° C. Phase C ingredients were post-added one at a time to Phase AB and the resulting mixture was stirred. The composition was then poured into a suitable container, labeled, and equilibrated to room temperature. Suitable equipment may include, for example, beakers equipped with an IKA digital overhead mixer equipped with a propeller blade, IKA RET control vise magnetic stir plates with heating probes, and IKA T25 Ultra Turrax homogenizers (all manufactured by IKA, Wilmington, NC, US).

TABLE 1

| | | Exemplary | Exemplary | | |
|---|---|---|---|---|---|
| Phase | Ingredient | Composition 1 | Composition 2 | Comparison 1 | Comparison 2 |
| A | USP Purified | Balance | Balance | 0 | 0 |
| | Water | | | | |
| A | Petrolatum | 0 | 0 | 0 | Balance |
| A | Polyol: glycerin | 30 | 10 | 0 | 0 |
| A | Emulsifier A¹ | 0.5 | 0 | 0 | 0 |
| A | Emulsifier B² | 0 | 0.25 | 0 | 0 |
| A | pH adjuster: Citric | 0.01 | 0 | 0 | 0 |
| | acid | | | | |
| A | Preservative³ | 1.0 | 0 | 0 | 0 |
| B | Emulsifier C⁴ | 0.5 | 0 | 0 | 0 |
| B | Emulsifier D⁵ | 0 | 0.25 | 0 | 0 |
| B | Emollient A⁶ | 37 | 65 | Balance | 0 |
| B | Emollient B⁷ | 7.5 | 0 | 0 | 0 |
| B | Emollient C⁸ | 0.5 | 0 | 0 | 0 |
| B | Emollient D⁹ | 0 | 0.5 | 0 | 0 |
| B | Structurant: | 2 | 0 | 0 | 0 |
| | glyceryl stearate | | | | |
| B | Structurant: | 0 | 0 | 27 | 0 |
| | beeswax | | | | |
| B | Tocopherol | 0 | 0.1 | 0.1 | 0.1 |
| | (Vitamin E) | | | | |
| C | Essential oil | 4 | 4 | 4 | 4 |
| | blend¹⁰ | | | | |
| C | Fixative: | 1.5 | 1.5 | 0 | 0 |
| | Polycitronellol | | | | |

Ingredients for Table 1
¹Potassium cetyl phosphate
²Sucrose laurate
³Propanediol and 1,2 Hexanediol
⁴Candelila/jojoba/rice bran polyglyceryl-3 esters and polyglyceryl-6 distearate
⁵Sucrose palmitate and sucrose stearate (Sucrablend ® SPV2)
⁶Caprylic/Capric triglycerides
⁷Hydrogenated vegetable oil
⁸Olea Europaea (Olive) fruit oil
⁹Cocos Nucifera (Coconut) oil
¹⁰Lavender Oil, Eucalyptus Oil, Rosemary Oil, Spearmint Oil, Wintergreen Oil Blend

Exemplary Compositions 1 and 2 shown above are examples of compositions containing different combinations of water, polyol component, emollient, olfactory component (essential oil blend), olfactory component fixative, and structurant. Comparison Compositions 1 and 2 shown above are examples of typical vapor compositions on the market. The viscosity of Exemplary Composition 1 was measured according to the Viscosity Test Method described herein and was 90,000-300,000 cP. The pH of Exemplary Composition 1 was measured according to the pH Test Method described herein and was 4.8-5.5.

In summary, personal care vapor compositions have been described which are oil/water emulsions and which include one or more polyols, one or more fatty acid triglycerides as emollients, an olfactory component which can comprise an essential oil blend, an olfactory component fixative, and in some embodiments a structurant. The personal care vapor compositions are ideal for the application to the skin of infants, have a high perception of vapor intensity and may be washed out of typical articles of clothing using consumer washing machines and detergents.

While examples of the personal care vapor compositions have been described above, the present invention is not limited thereto but only by the scope and spirit of the appended claims.

We claim:

1. A personal care vapor composition, comprising:
at least about 1 percent of a polyol component selected from one or more C₃-C₆ polyols;
at least about 20 percent of an emollient selected from one or more triglycerides of C₆-C₁₈ fatty acids, isopropyl isostearate and mixtures thereof;
from about 0.5 to about 10 percent of an olfactory component comprising one or more essential oils;
an olfactory component fixative selected from the group consisting of amphiphilic terpene-based polyethers, Commiphora myrhha extract, methyl hydrogenated rosinate and mixtures thereof, the olfactory component fixative being present in the personal care vapor composition at a ratio of 1:1 to 1:4 relative to the olfactory component; and
water, wherein the personal care vapor composition has an aqueous phase and an oil phase, wherein the aqueous phase includes the water and the polyol component, wherein the oil phase includes the emollient and the olfactory component, and wherein the oil phase is at least 30 percent of the personal care vapor composition.

2. The composition of claim 1, wherein the polyol component is selected from the group consisting of glycerin, diglycerin, erythritol, arabitol, xylitol, mannitol, sorbitol, galactitol, fucitol, maltilol, mannose, inositol, triethyleneglycol, sodium pyrrolidone carboxylic acid (PCA), zinc PCA, propylene glycol, pentylene glycol, butylene glycol and mixtures thereof.

3. The composition of claim 1, wherein the polyol component is present in the composition at no more than about 50 percent of the composition.

4. The composition of claim 3, wherein the polyol component is present in the composition in the range of about five to about thirty percent.

5. The composition of claim 1, wherein the emollient is selected from the group consisting of isopropyl isostearate, caprylic/capric triglycerides, one or more vegetable oils and mixtures thereof.

6. The composition of claim 1, wherein the emollient is present in the composition at no more than about seventy percent.

7. The composition of claim 6, wherein the emollient is present in the composition within a range of about thirty to about seventy percent.

8. The composition of claim 1, wherein the olfactory component is selected from the group consisting of eucalyptus oil, lavender oil, rosemary oil, wintergreen oil, spearmint oil and mixtures thereof.

9. The composition of claim 1, wherein the olfactory component is present in the composition within a range of about one percent to about four percent of the composition.

10. The composition of claim 9, wherein the olfactory component is about two percent of the composition.

11. The composition of claim 1, wherein the olfactory component fixative is present in the personal care vapor composition at a ratio of about 1:2 to about 1:4 relative to the olfactory component.

12. The composition of claim 1, wherein the olfactory component fixative is polycitronellol.

13. The composition of claim 1, further comprising from about 0.01% to about 15% of a structuring agent selected from the group consisting of glyceryl stearate, stearic acid, palmitic acid, stearyl alcohol, cetyl alcohol, behenyl alcohol, one or more polyethylene glycol ethers of stearyl alcohol having an average of about 1 to about 5 ethylene oxide units, one or more polyethylene glycol ethers of cetyl alcohol having an average of about 1 to about 5 ethylene oxide units, and mixtures thereof.

14. The composition of claim 1, further comprising from about 0.05% to about 5.0% of an emulsifier selected from the group consisting of fatty phosphate monoesters, fatty acid sucrose esters, phospholipids, fatty acid mono- and diglycerides, ethoxylated alcohols, polysorbates and mixtures thereof.

15. A personal care vapor composition, comprising:
at least about ten percent of a polyol component selected from one or more water-soluble polyols;
water, a ratio of the water to the polyol component being in the range of about 2.0 to about 0.5;
at least about twenty percent of an emollient selected from the group consisting of triglycerides of C₆-C₁₈ fatty acids, isopropyl isostearate and mixtures thereof;
from about 1 to about 10 percent of one or more essential oils; and
an essential oil fixative selected from the group consisting of amphiphilic terpene based polyethers, Commiphora myrhha resin extract, methyl hydroxylated rosinate and mixtures thereof, a ratio of the essential oil fixative to the one or more essential oils being in the range of about 1:1 to about 1:4.

16. The composition of claim 15, wherein the polyol component is selected from the group consisting of polyhydric alcohols, 1,3 butylene glycol, propylene glycol, pentylene glycol, glycerin, diglycerin, polyethylene glycol ethers of glycerin, glycerol monopropoxylate, glycogen, hexylene glycol, hydrogenated starch hydrolysates, hydrolyzed mucopolysaccharides, glycosaminoglycans, methoxy PEG-10, methyl gluceth-10, methyl gluceth-20, methyl glucose, 3-methyl 1,3 butanediol, polyethylene glycol, PEG-15 butanediol, PEG-4, PEG-5 pentaerythritol, PEG-6, PEG-8, PEG-9, 1,2 pentanediol, PPG-1 glyceryl ether, PPG-9, 2-pyrrolidone-5-carboxylic acid and salts thereof, saccharide isomerate, sericin, silk amino acids, sodium acetylhyaluronate, sodium hyaluronate, sodium polyaspartate, sodium polyglutamate, sorbet 20, sorbet 6, glucose, mannose, polyglycerol sorbitol, trehalose, triglycerol, trimethoylpropane, tris (hydroxymethyl) amino methane salts, yeast extract and mixtures thereof.

17. The composition of claim 15, wherein the polyol component is present in the composition in the range of about five to about thirty percent.

18. The composition of claim 15, wherein the emollient is selected from the group consisting of isopropyl isostearate, caprylic/capric triglycerides, one or more vegetable oils and mixtures thereof.

19. The composition of claim 15, wherein the emollient is present in the composition in a range of about 30 to about 70 percent.

20. The composition of claim 15, wherein the essential oil is selected from the group consisting of eucalyptus oil, lavender oil, rosemary oil, spearmint oil, wintergreen oil, cedar leaf oil, cedar wood oil, turpentine, thymol, anethole, coriander, mandarin, petitgrain, armoise, cumin nitrile, marjoram sweet, pink pepper, basil, frankincense, methyl salicylate, bergamot, galbanum, neroli, Sichuan pepper, black pepper, grapefruit, nutmeg oil, tea tree oil, cardamom, jasmine, oil of black pepper, verveine, chinese ginger oil, orange sweet, vetivert, cinnamon leaf, lavendine, palmerosa, violet leaves, clary sage, lemongrass, patchouli oil, ylang, clove, lime, olbus oil and mixtures thereof.

21. The composition of claim 15, wherein the essential oil is about two to about four percent of the composition.

22. The composition of claim 15, wherein the essential oil fixative is polycitronellol.

23. The composition of claim 15, further comprising from about 0.01% to about 15% of a structuring agent selected from the group consisting of glyceryl stearate, stearic acid, palmitic acid, stearyl alcohol, cetyl alcohol, behenyl alcohol, one or more polyethylene glycol ethers of stearyl alcohol having an average of about 1 to about 5 ethylene oxide units, one or more polyethylene glycol ethers of cetyl alcohol having an average of about 1 to about 5 ethylene oxide units, and mixtures thereof.

24. The composition of claim 15, further comprising from about 0.05% to about 5.0% of an emulsifier selected from the group consisting of fatty phosphate monoesters, fatty acid sucrose esters, phospholipids, fatty acid mono- and diglycerides, ethoxylated alcohols, polysorbates and mixtures thereof.

25. A method of using a personal care vapor composition, the method comprising the steps of:
supplying a personal care vapor composition comprising water, one or more water-soluble polyols, one or more triglycerides of C₆-C₁₈ fatty acids, an olfactory component, and an olfactory component fixative;
applying the personal care vapor composition to the skin of a human;
contacting a fabric to the skin of the human in an area where the personal care vapor composition has been applied, whereupon some of the personal care vapor composition adheres to the fabric;
removing the fabric from the human;
washing the fabric in a washing machine using a cold water cycle and a detergent; and
responsive to the step of washing the fabric, removing the personal care vapor composition from the fabric.

26. The method of claim 25, wherein the step of contacting the fabric to the skin of the human comprises the step of leaving the fabric in contact with the personal care vapor composition for at least eight hours.

27. The method of claim 25, wherein the personal care vapor composition further includes an emulsifier selected from the group consisting of fatty phosphate monoesters and fatty acid sucrose esters.

28. A method of using a personal care vapor composition, the method comprising the steps of:
supplying a personal care vapor composition comprising water, a polyol component, at least one emollient, an olfactory component and an olfactory component fixative; and
applying a safe and effective amount of the personal care vapor composition to at least a portion of the chest, back and/or neck of a human in need thereof.

29. The method of claim 28, wherein the polyol component comprises one or more C₃-C₆ polyols.

30. The method of claim 28, wherein the at least one emollient is selected from the group consisting of triglycerides of C₆-C₁₈ fatty acids, isopropyl isostearate and mixtures thereof.

31. The method of claim 28, wherein the olfactory component fixative is polycitronellol.

32. The method of claim 28, wherein the personal care vapor composition further includes a structurant selected from the group consisting of glyceryl stearate, stearic acid, palmitic acid, stearyl alcohol, cetyl alcohol, behenyl alcohol, one or more polyethylene glycol ethers of stearyl alcohol having an average of about 1 to about 5 ethylene oxide units, one or more polyethylene glycol ethers of cetyl alcohol having an average of about 1 to about 5 ethylene oxide units, and mixtures thereof.
